(19) United States
(12) Patent Application Publication
(10) Pub. No.: US 2023/0414876 A1
(43) Pub. Date: Dec. 28, 2023

(54) MEDICAL NEEDLE-FREE INJECTION SYSTEM AND METHOD

(71) Applicant: INTEGRIMEDICAL LLC, Bainbridge, IN (US)

(72) Inventors: Hassan Salari, Irvine, CA (US); Ankur Naik, Minneapolis, MN (US); Sambath Kumar Rajagopal, Tamil Nadu (IN)

(21) Appl. No.: 18/244,868

(22) Filed: Sep. 11, 2023

Related U.S. Application Data
(63) Continuation-in-part of application No. 17/123,968, filed on Dec. 16, 2020, now Pat. No. 11,752,269.
(60) Provisional application No. 62/949,984, filed on Dec. 18, 2019.

(51) Int. Cl. A61M 5/20; A61M 5/315

(52) U.S. Cl. CPC A61M 5/2033; A61M 5/31511; A61M 5/31513

(57) ABSTRACT

A spring-loadable fluid injection device for a needle-free syringe is provided and comprises a trigger positioned within the housing and actuated by depression of a push button or lever disposed opposite the dispensing end at an external actuation surface. The trigger has a trigger latch configured to release a trigger catch as heaving part of the spring-loaded piston of the device. The trigger catch is configured to urge the trigger latch in a direction transverse to the injection axis when the piston moves from the unloaded position to the loaded position. The compression spring is retained around the piston and retained within the housing by a piston head. The device further comprises a syringe adapter and a safety cap which prevents accidental triggering. An embodiment further includes a prefilled syringe adapter for aseptic filling of a needle-free syringe from a prefilled syringe.

### CROSS-REFERENCE TO RELATED APPLICATIONS

This application is a continuation-in-part application of co-pending U.S. patent application Ser. No. 17/123,968, filed Dec. 16, 2020, which claims priority pursuant to 35 U.S.C. 119(e) to U.S. Provisional Patent Application Ser. No. 62/949,984, filed Dec. 18, 2019, the entire disclosures of which are incorporated herein by reference.

### FIELD OF THE INVENTION

The present invention relates to a medical injection device and kit. More particularly, the present invention relates to an improved spring-loaded needle-free hypodermic injection device.

### BACKGROUND OF THE INVENTION

Medical needle-free injection technologies deliver medication to a subject without piercing the subject's skin with a conventional needle. Some benefits of needle-free injection compared to conventional injections include reduced risk of needle stick accidents; eliminated risk of disease transmission from re-used needles; reduced bleeding or bruising; faster and more reproducible drug delivery; avoidance of emotional distress from needle-phobia; and being better suited to self-administration. Depending on its specifications, a needle-free injection device may be suitable for subcutaneous, transdermal, and/or intramuscular use.

Injection free devices inject liquid or powdered medicine through micro-orifices by rapidly applied high pressure. Coil springs, gas-springs, and/or explosives supply the rapid high pressure. Improvements to such devices continue to be developed, making them safer to use by healthcare professionals and patients.

For instance, Thai et al. disclosed in PCT patent application WO2012154025 A2 a spring-powered needle-free hypodermic injector. The spring is associated with a piston head, both of which are slidably housed within the handle of the injector. When compressed, the spring is positioned in the handle opposite the open end of the device, and the open end is connected to the plunger end of a needle-free syringe that holds the fluid to be injected. The compressed spring is held in place by the stopper of a trigger which is pivotally mounted to the handle near the opening of the device. When the trigger is actuated by displacing the trigger stopper from the piston head, the power stored in the spring drives the piston head to depress the plunger of the syringe and force fluid from the nozzle into the subject's skin. A return spring returns the trigger to the non-actuated position. A slidable safety lock on the handle near the opening of the device can be slid under an end of the trigger to prevent accidental actuation of the device. The injector disclosed by Thai et al., however, is limited by only being able to be used by medical professionals in a clinical setting.

Nzike et al. in U.S. Pat. No. 9,421,335 proposed a clutch mechanism for rotationally locking and releasing the dose member within a drive sleeve. The clutch, however, lacks positive safety against accidental triggering.

Most of the marketed needle-free injectors have serious limitations. For example, the placement of a trigger on the side of an injector may not be convenient for self-injection, as it may be difficult for the user to orient the injector perpendicular to the user's skin and hold it securely there with the user's thumb pointing toward the user's body. When an injector is not held securely and perpendicularly against the skin, it may fail or cause medication leakage during injection. In addition, the spring force of some injectors is only sufficient to inject a small volume of fluid. Small volume injections are not optimal for some users/patients. In some injectors, the form factor is fixed, with the result that the injector cannot be modified to increase its spring force for higher volume injections. As an alternative to using spring power, certain needle-free injectors use compressed gas, battery power, or a pyrotechnic charge to drive fluid through the nozzle and into or through the skin or tissue of the subject.

For example, Boyd et al. in U.S. Pat. No. 8,734,384 disclose a compressed gas injector which operates by pushing the piston into a fluid filled chamber, which pressurizes the fluid and creates a fluid jet as it empties through a jet orifice. Actuation of the injector in this device is preferably triggered from pressing the device against the patient's skin. Triggering the device however is not handy and the gas pressure pistons can be misused or mishandled and require resupply with limited shelf life.

Some needle-free injectors are intended for disposal after a single use, while others are reloadable. Certain other needle-free injectors inject powdered substances as micro projectiles.

Improvement on the state of the art is needed. More specifically, safer, and handier mechanisms are in demand which can provide precision metered doses at precision selected pressures for self-administering safety. Hand power reloading is preferred over prepackaged consumable power cartridges.

The object of the invention is to improve on the state of the art by overcoming the listed and other shortcomings in needle-free liquid medicine delivery into human and/or animal bodies.

### SUMMARY OF THE INVENTION

The present invention comprises a medical fluid injection device, system, and method. In an exemplary embodiment, a medical fluid injection device of the present invention comprises a housing having an inner surface and an outer surface which extend from a trigger end to a dispensing end along an injection axis, the trigger end comprising an external actuation surface; a syringe adapter removably or irremovably affixed to the dispensing end of the housing, the syringe adapter configured to retain a needle-free syringe such that a plunger of the needle-free syringe is movable within the housing along the injection axis; a piston retained within the housing by the syringe adapter and movable along the injection axis between a loaded position and an unloaded position, the piston comprising a piston head near the dispensing end and a trigger catch near the trigger end of the housing, the piston head being movably positioned to bias the plunger of the needle-free syringe along the injection axis; a compression spring retained around the piston by the piston head, the piston and compression spring sized relative to the housing such that the compression spring is compressible along the injection axis within the housing; and a trigger mounted within the housing near the trigger end and movable between a non-actuated position and an actuated position by depression of a push button or lever disposed at the external actuation surface of the housing. In an exemplary embodiment, the trigger assembly comprises a trigger latch configured to engage a trigger catch when the trigger is in the non-actuated position and the piston is in the loaded position and further configured to release the trigger catch when the trigger is in the actuated position, wherein the trigger catch is configured to urge the trigger latch in a direction approximately transverse to the injection axis when the piston moves from the unloaded position to the loaded position.

The trigger catch of some embodiments of the fluid injection device comprises an annular engagement surface extending radially outward from the injection axis and facing toward the dispensing end of the housing. The trigger latch of the piston of some embodiments comprises an engagement surface extending inwardly toward the injection axis and facing toward the trigger end of the housing, and which engages the annular engagement surface of the trigger catch when the trigger is in the non-actuated position and clears the trigger catch when the trigger is in the actuated position. In other embodiments, the trigger catch of the fluid injection device further comprises an annular beveled surface which faces away from the injection axis towards the trigger end of the housing. In such embodiments, the trigger latch further comprises a beveled surface which faces toward the injection axis and toward the dispensing end of the housing.

In an exemplary embodiment, the trigger assembly comprises a lever biased toward a push button or lever trigger by a return spring disposed within the housing. The return spring in some embodiments is a clip spring biased against the lever of the trigger. In other embodiments, the return spring is a coiled torsion spring or other type of spring.

The fluid injection device of some embodiments further comprises a movable safety cap for blocking depression of the push button or trigger lever. The safety cap of some such embodiments is pivotally mounted to the external actuation surface of the housing and configured for being popped up by the thumb or fingernail of a self-injecting patient.

The interior surface of some embodiments of the housing of the fluid injection device comprises a shelf extending inward toward the injection axis, forming a narrower tube toward the trigger end of the fluid injection device, and configured for blocking the compression spring from contacting the trigger. In embodiments, the combination of the shelf and the piston head accommodate compression of the spring within the housing of the fluid injection device. In an exemplary embodiment, the piston of the present invention includes a bevel which extends into the narrower tube of the fluid injection device when the piston is in loaded position. Such bevel of the piston allows for easier and quieter loading of the piston and quieter actuation of the device and release of the piston.

The syringe adapter of the fluid injection device of some embodiments comprises an outer surface that extends away from the injection axis beyond the outer surface of the housing. In an exemplary embodiment, the syringe adapter comprises an outer surface that extends away from the injection axis beyond the outer surface of the housing and tapers from a wider syringe adapter outer surface proximal end to a narrower syringe adapter outer surface dispensing end. The syringe adapter of some embodiments is removable.

In some embodiments, the housing is configured to removably accept alternatively shaped or configured syringe adaptors. In some such embodiments, different syringe adapters are configured to be used with different types and/or sizes of syringes.

In some embodiments of the present invention, the fluid injection device is configured such that the piston and compression spring can be removed from the housing when the syringe adapter is removed from the housing. The housing of some embodiments is configured to be able to removably accept one or both of alternatively sized pistons and springs.

In an exemplary embodiment of the present invention, a fluid injection system further comprises a loader configured for receiving the fluid injection device and for loading the fluid injection device from an unloaded position to a loaded position. In some embodiments, the fluid injection device and the loader of the present invention are provided together as a kit.

In an exemplary embodiment, the loader of the present invention is configured to receive the fluid injection device on an interior retaining surface, wherein the loader is movable about a hinge between an open state and a closed state to extend a plunger into an open end of the syringe adapter to contact the piston head so as to push the piston from the unloaded position into the loaded position. In an exemplary embodiment, the interior retaining surface comprises a fluid injection device receiver connected to a syringe adapter receiver, the syringe adapter receiver including a lip adapted to engage the outer surface of the syringe adapter of the fluid injection device. In an exemplary embodiment, the fluid injection device receiver is shaped to receive and closely fit around the body of an inserted fluid injection device so as to prevent side to side movement when loading the piston. In an exemplary embodiment, the syringe adapter receiver is shaped to receive and closely fit around an inserted syringe adapter so as to prevent side to side movement when loading the piston.

In some embodiments, the syringe adapter of the fluid injection device is removable. In some embodiments, a kit of the present invention further comprises one or more additional compression springs, each of which is distinctly configured to provide a different spring force. In some embodiments, the syringe adapter contains features that allow the syringe adapter to work smoothly with springs designed for the appropriate size of the syringe adapter. In some embodiments, the kit further comprises one or more additional syringe adapters, each of which is configured to retain a different needle-free syringe.

In an exemplary embodiment of the present invention, the engagement trigger comprises a lever mechanism having a lever trigger engageable by a user about a pivot point on one side of the pivot point. In an exemplary embodiment of the present invention, when engaged, an internal extension of the engagement trigger mechanism positioned about the pivot point opposite the lever trigger is configured to apply pressure to an internal, spring-loaded lever connected about an internal pivot point to a piston engagement latch configured to engage with a catch portion of a piston and hold the piston in a non-actuated position when the piston is loaded.

In an exemplary embodiment of the present invention, the lever trigger pivot point is positioned at the trigger end of the fluid injection device near the internal return spring and a second, internal spring-loaded lever. In an embodiment, the engageable portion of the lever trigger extends outward from the pivot point and configured for actuation by a user. In an embodiment, the lever trigger extends outward and upward approximately 45-degrees from the pivot point in a loaded, non-actuated position. Such lever trigger assembly design accommodates easier actuation of the trigger compared to a spring-loaded button because it requires less force to actuate a lever compared to a button. Moreover, such lever trigger assembly allows for utilization of greater spring force compared to prior art needleless injector devices while maintaining an easily engageable trigger for the average user.

In an exemplary embodiment of the present invention, the sound directing construction and lever trigger assembly allow for the present fluid injection device to be easily actuated by laypersons with minimal intimidation. Such device design accommodates use of powerful internal spring force for increased consistency and higher volume injections.

In an exemplary embodiment, the present fluid injection device is used in combination with a needle-free syringe configured for attachment to the syringe adapter and configured for sealing the dispensing end of the fluid injection device. In an exemplary embodiment, the needle-free syringe includes a series of holes small enough for fluid not to leak without application of pressure but large enough to dispense fluid therethrough with force applied to the plunger by the piston of the present injector device, creating pressure within the needle-free syringe.

In an exemplary embodiment, the present fluid injection device and system includes one or more adapters for use to aseptically fill a needle-free syringe of the present system with a fluid from a prefilled syringe.

In an exemplary embodiment, prefilled syringe adapters of the present invention include luer lock connectors for connection to prefilled luer lock syringes containing fluid to be injected. In an exemplary embodiment, prefilled syringe adapters of the present invention accommodate filling needle-free syringes with fluid from prefilled syringes having needles.

In embodiments, the needle-free syringes and/or syringe adapter(s) of the present invention include male and female threaded connectors or other types connectors. In further embodiments, the syringes and syringe adapter of the present invention include luer lock connectors for connection to one another.

In an exemplary embodiment, the present fluid injection device provides increased consistency, provides deeper penetration of fluid into a patient's body, and allows for use for higher volume injections compared to prior art devices.

In an exemplary embodiment, the present fluid injection system further comprises adapters for connection to vials containing fluid to be injected and/or adapters for connection between a syringe adapter of the present invention and syringes. In embodiments, the fluid injection system further comprises prefilled needle-free syringes configured for use with the fluid injection device.

In embodiments, the present system includes sterile packaging for syringes, vials, and/or adapters.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

A preferred embodiment of the invention, illustrative of the best mode in which the applicant has contemplated applying the principles, is set forth in the following description and is shown in the drawings and is particularly and distinctly pointed out and set forth in the appended claims.

FIG. 1 shows an isometric view of a fluid injection device in accordance with an embodiment of the present invention, in a loaded state and attached to a needle-free syringe.

FIG. 2 shows an offset sectional view of the fluid injection device of FIG. 1, in a loaded and non-actuated state.

FIG. 3A shows a full sectional view of a portion of the fluid injection device of FIG. 1, in a loaded and non-actuated state.

FIG. 3B shows a full sectional view of the fluid injection device of FIG. 1, in an unloaded and non-actuated state.

FIG. 4A shows an isometric view of an open loader retaining the fluid injection device of FIG. 1.

FIG. 4B shows a partially exploded view of the open loader of FIG. 4A without the fluid injection device assembled compact.

FIG. 4C shows a partially exploded view of the open loader of FIG. 4A without the fluid injection device disassembled and stripped apart.

FIG. 5 shows a front, elevational view of an embodiment of a fluid injection device of the present invention.

FIG. 6 shows a perspective, cross-sectional, exploded view of the fluid injection device.

FIG. 7 shows an elevational, cross-sectional view of the fluid injection device.

FIG. 8 shows an enlarged, elevational, cross-sectional view of a trigger assembly of the fluid injection device.

FIG. 9 shows an upper, perspective view of a prefilled syringe adapter piece of the present invention.

FIG. 10 shows an elevational view of the prefilled syringe adapter piece.

FIG. 11 shows a cross-sectional, elevational view of the prefilled syringe adapter piece.

FIG. 12 shows a top, plan view of the prefilled syringe adapter piece.

FIG. 13 shows a bottom, plan view of the prefilled syringe adapter piece.

FIG. 14 shows a cross-sectional, elevational view of the prefilled syringe adapter piece.

FIG. 15 shows a top, plan view of the prefilled syringe adapter piece.

FIG. 16 shows a cross-sectional, elevational view of the prefilled syringe adapter piece.

FIG. 17 shows a further elevational view of the prefilled syringe adapter piece.

FIG. 18 shows a further cross-sectional, elevational view of the prefilled syringe adapter piece.

FIG. 19 shows a side, perspective, exploded view of the prefilled syringe adapter piece.

FIG. 20 shows an upper, perspective, exploded view of the prefilled syringe adapter piece and sterile packaging.

FIG. 21 shows a bottom, plan view of sterile packaging for the prefilled syringe adapter piece of the present invention.

FIG. 22 shows a side, elevational view of sterile packaging for the prefilled syringe adapter piece.

The drawing figures do not limit the present invention to the specific embodiments disclosed and described herein. The drawings are not necessarily to scale, emphasis instead being placed upon illustrating the principles of the invention.

### DETAILED DESCRIPTION OF THE PREFERRED EMBODIMENTS

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

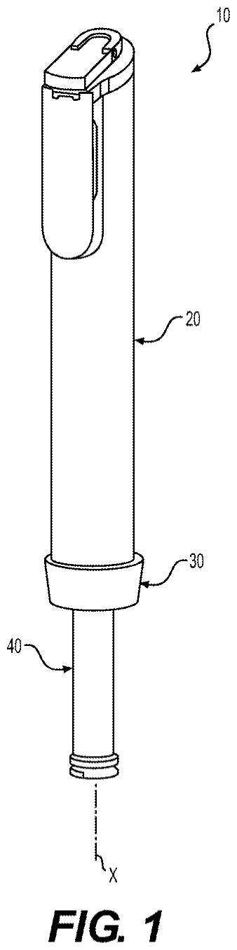

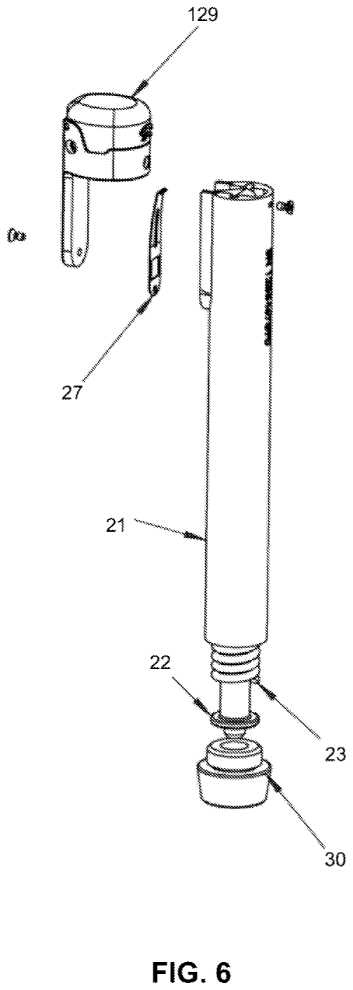

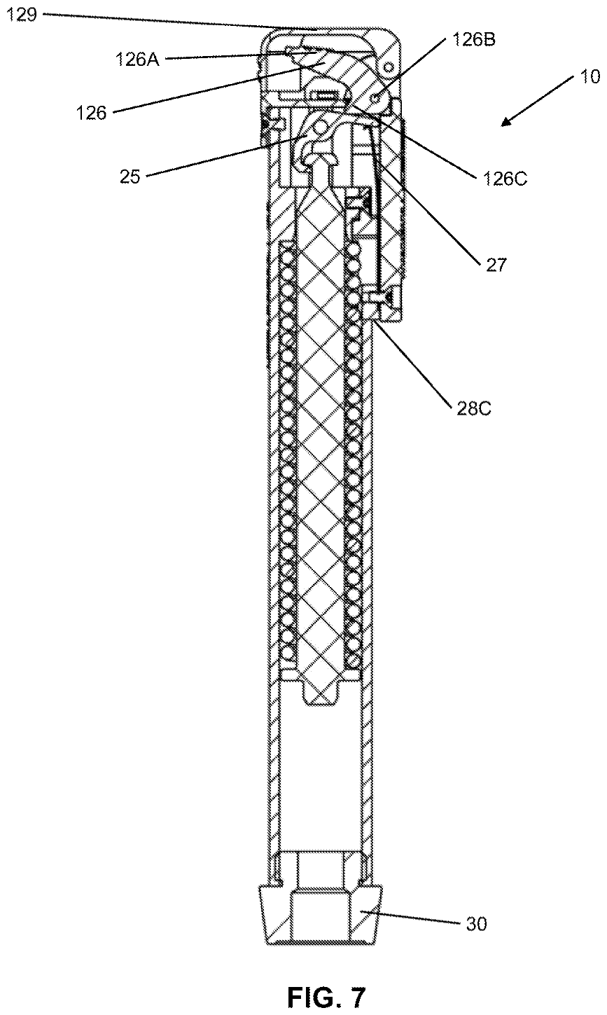

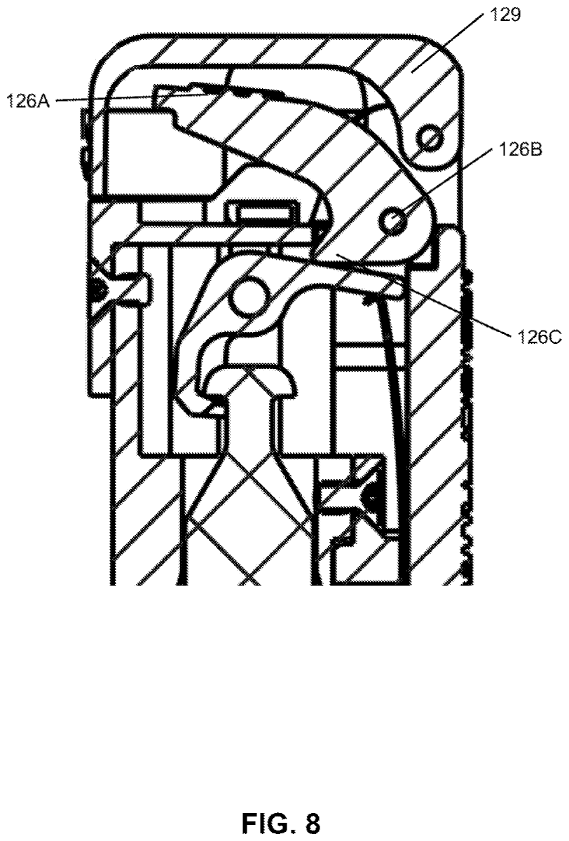

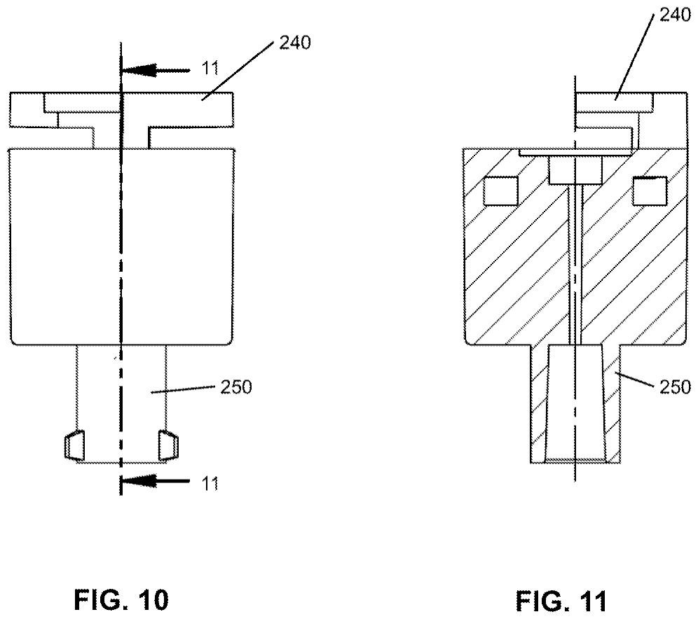

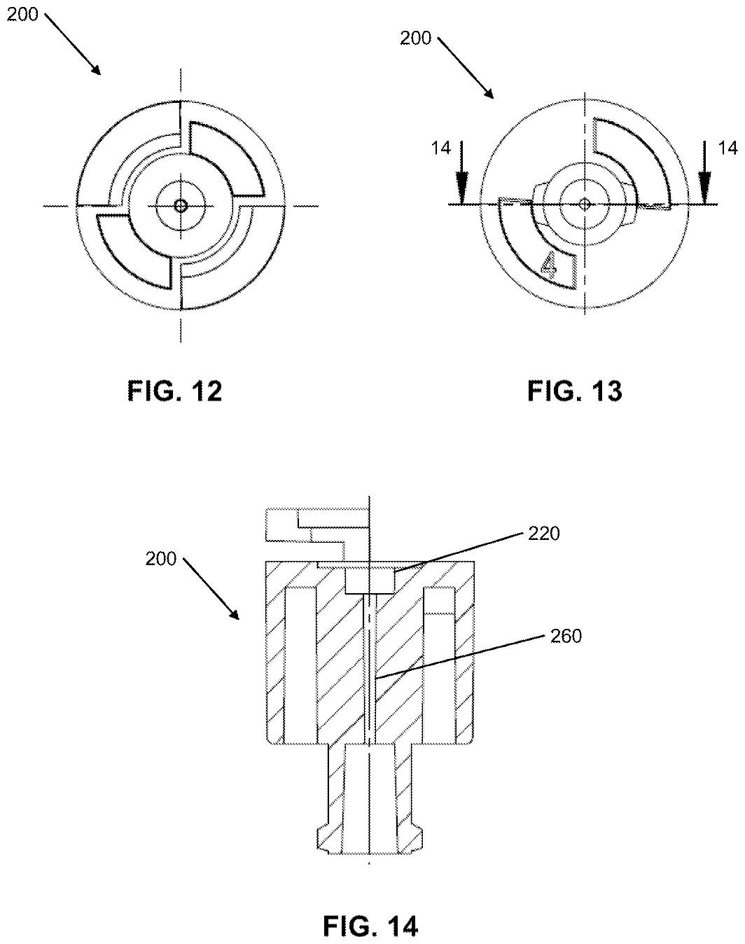

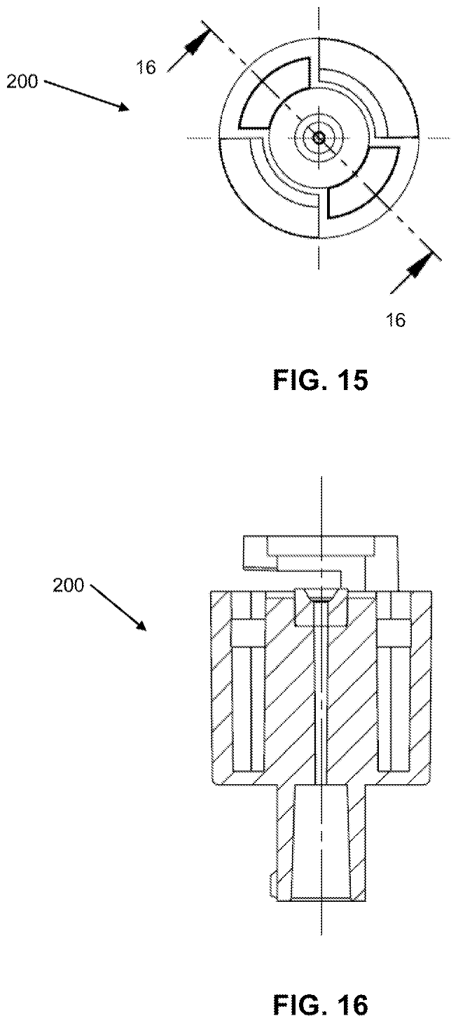

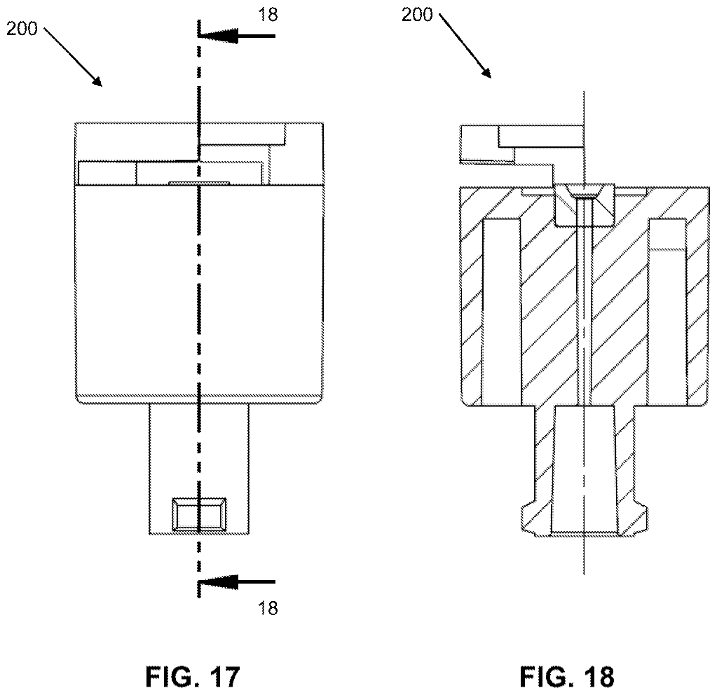

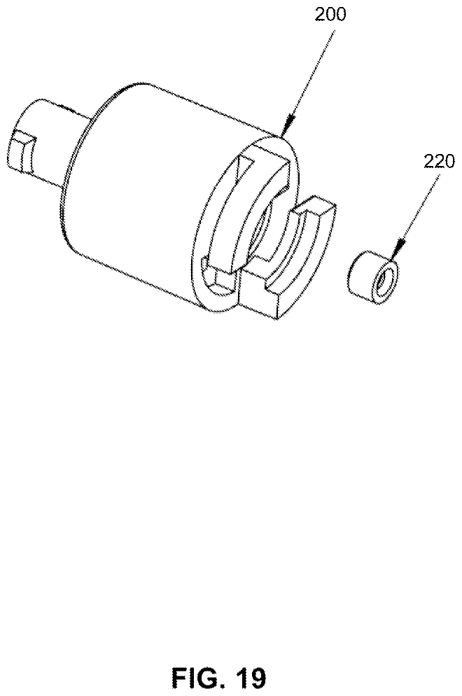

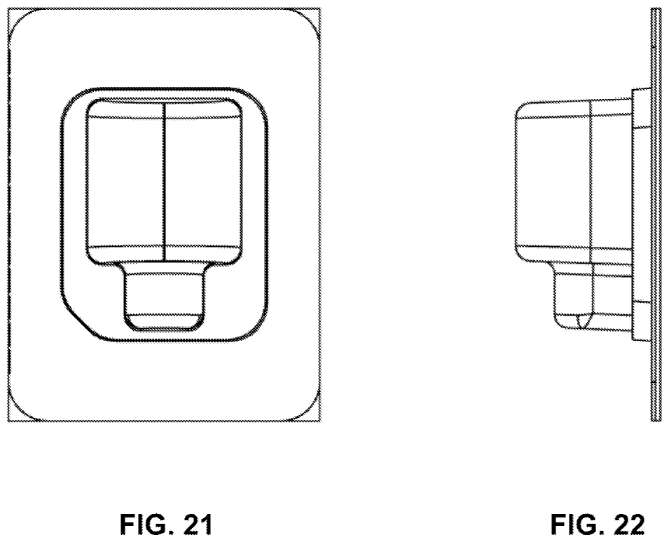

The following description discloses a fluid injector device for use with a needle-free syringe, with exemplary embodiments shown in FIGS. 1-22. Attention is now turned to FIG. 1, which illustrates a fluid injector device 10 comprising a housing 20 with a triggered spring-loaded mechanism, a needle-free syringe adapter 30, and a needle-free syringe 40, whereas the injector 10, housing 20, syringe adapter 30, and syringe 40 are all aligned along a dispensing or injection axis X. In an exemplary embodiment of the present invention, the fluid injector device 10 has the same outer appearance in both its loaded and unloaded states. Nevertheless, without reloading, triggering does not cause a secondary injection.

The housing and other components of the fluid injector device of various embodiments are made of stainless steel, for example, or any other suitable metal (e.g., zinc/chrome-plated carbon steel) or any other suitable rigid material, such as a rigid plastic (e.g., PC/ABS plastic) or the like. In some embodiments, the housing includes a generally continuous cylindrical metal (e.g., stainless steel, zinc, carbon, etc.) material tube inner and outer surfaces with openings only at the trigger end and the dispensing end. In some such embodiments, the only non-metal components enclosing the housing are located at the openings at the trigger end (e.g., the venting slot 28C, push button 26, clip spring 27, housing top retainer 28, and safety cap 29), and the dispensing end (e.g., the syringe). The wall thickness of the metal utilized for the housing is sufficient to support the internal components of the injector (e.g., spring, piston, etc.) and also to prevent significant sound created within the housing (e.g., from the spring/piston actuation) from escaping the sidewalls. This structure and the heavier and thicker metal material of the housing result in limiting the amount of sound that is produced by the injector during operation. The syringe itself functions to seal the dispensing end opening and absorb sound produced by the actuation mechanism (e.g., spring, etc.) during actuation. As such, the only point in which sound generated during actuation can travel outside of the housing is through the non-metal components at the trigger end (which in some embodiments is sealed by the non-metal components such as the push button or lever, housing top retainer, safety cap, etc.). In addition to reducing the sound generated, the sound is directed away from the subject being injected and is less likely to startle the subject during injection.

Embodiments of the housing include a variety of sizes depending on its intended manner of use. For example, the housing of some embodiments is sized and shaped to be held comfortably by the user in one hand (e.g., a cylinder of approximate dimensions 13 cm long×1.5 cm diameter). The user may be someone who is self-injecting, for example in the abdomen, arm, leg or another location of the body. Alternatively, the user may be a medical professional or technician who is injecting a human or animal subject.

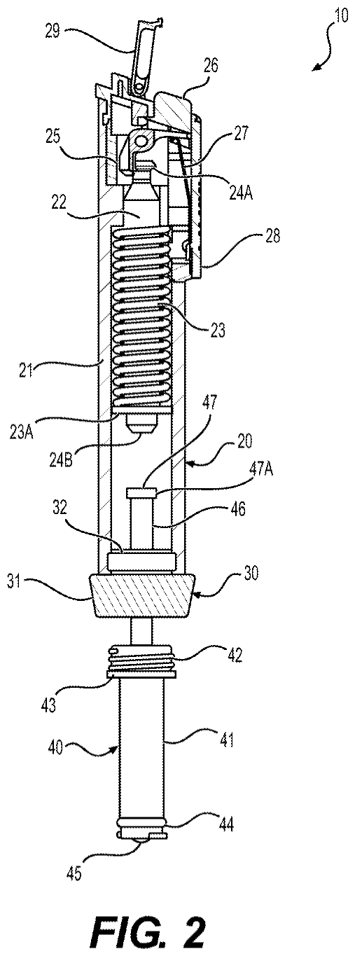

In an exemplary embodiment, as shown in FIG. 2, the housing 20 comprises a body 21, a piston 22 with a trigger catch shoulder 24A and hammer tip 24B, a piston stop 23A, coil spring 23, trigger catch 25, push button 26, clip spring 27, housing top retainer 28, venting slot 28C, and safety cap 29.

The syringe adapter 30 of the present invention comprises a threaded socket 31 and a threaded shoulder 32.

In an exemplary embodiment, the needle-free syringe 40 utilized with the present invention comprises a liquid medicine dispenser capsule 41, a threaded head 42, a head stop shoulder 43, a dispensing tip 44, and an injection nozzle 45 with one or more orifices. In an exemplary embodiment of the present invention, the injection nozzle orifices of the needle-free syringe are larger than conventional, which provides greater penetration with faster force and less constriction compared to prior art devices. In an exemplary embodiment of the present invention, the injection nozzle orifices have a diameter ranging from 0.17 mm to 0.23 mm. In embodiments the injection nozzle orifices have a diameter ranging from 0.15 mm to 0.25 mm. In further embodiments, the injection nozzle orifices have a diameter ranging from 0.10 mm to 0.30 mm.

The trigger end of the housing includes an external actuation surface disposed transverse to the injection axis X. This places the actuation surface in good position for the thumb of a user to actuate the device for self-injection by depression of a push button or lever disposed at the external actuation surface. For example, the push button or lever of some embodiments is configured to be depressed in line or substantially in line with the injection axis. Some embodiments of the fluid injector device further comprise a movable safety cap for blocking depression of the push button or lever. The safety cap of various embodiments is capable of being mounted by any suitable way. For example, the safety cap of some embodiments is capable of being pivotally mounted to the external actuation surface of the housing so that it is capable of being easily lifted (e.g., with a thumb) to access the push button or lever when necessary and, just as easily, reseated (e.g., with a thumb or with the thumb fingernail tip) to block accidental actuation. The pivotal mount of some embodiments comprises a metal pin or in other embodiments is made of a flexible plastic or any other type of hinge. The safety cap of some embodiments is alternatively mounted by non-hinged means, e.g., via a removable friction fit or the like. Teetering of the cap (such that the cap is designed to fall back over the push-button through gravity), to prevent cap loss, is capable of being used for any removable cap. Teetering of the cap also improves safety by preventing accidental activation of the push button or lever if the cap were left removed or lost.

The dispensing end of the housing is connected to a syringe adapter configured to retain the needle-free syringe such that the plunger of the needle-free syringe is movable within the housing along the injection axis X. For example, in some embodiments, threading within the open end of the syringe adapter mates with the housing of the needle-free syringe. In certain embodiments, the threading consists of 3 threads. In other embodiments, the threading consists of 2, 3, 4, 5, 6, 7, 8 or more threads. The threading in some embodiments is configured to mate with commercially available syringes.

In an exemplary embodiment of the present invention, the syringe adapter is affixed to the dispensing end of the housing. In certain embodiments, the syringe adapter is removably affixed. For example, the syringe adapter of some embodiments is secured by threading on an angular position which mates with threading on the inner surface of the housing. In alternative embodiments, the syringe adapter is not removable or comprises part of the housing. In embodiments, the syringe adapter comprises a rounded outer surface that extends away from the injection axis beyond the outer surface of the housing, tapering from a wider proximal end to a narrow dispensing, or distal, end.

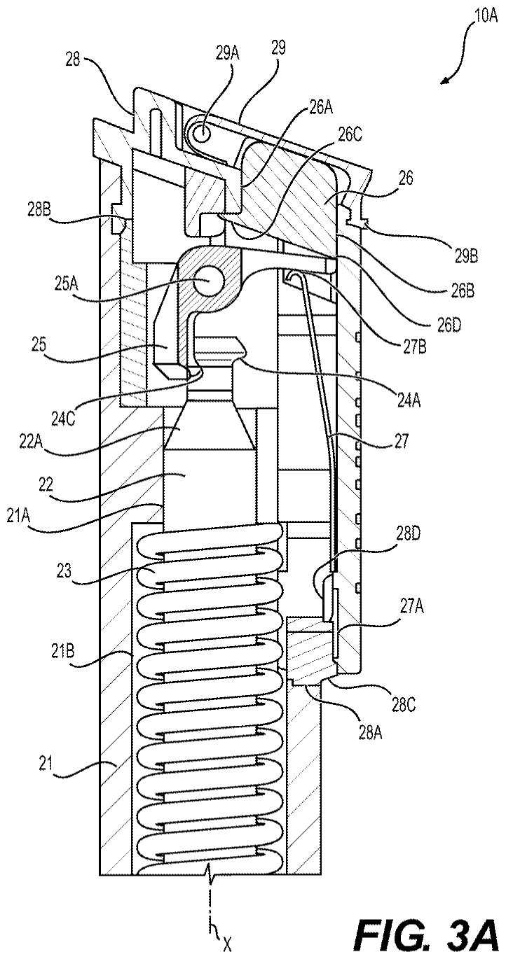

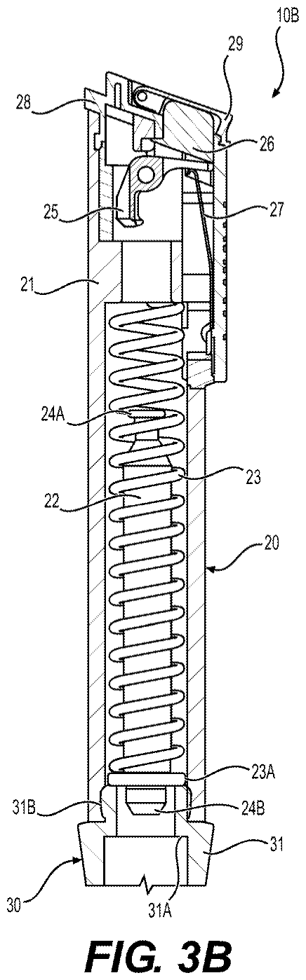

Attention is now turned to an embodiment of a triggering mechanism of the present invention. FIG. 3A, in isometric partial section view, illustrates an embodiment of the injector head 10A with the trigger mechanism. The injector head 10A comprises housing body 21 with inner surface 21A, which guides the spring 23, which guides the piston 22 having a neck 22A with a beveled trigger catch shoulder 24A, a trigger latch 25 pivoting on a pin 25A and catching at a contact point 24C, a push button 26 with a front sliding guide 26A, a rear sliding guide 26B, a restrainer protrusion 26C, and a lever contact 26D; a button position restoring clip spring 27 with an end fixture 27A and sliding tip 27B; a housing top retainer 28 with a lower stop socket 28A, upper pop-in socket 28B, and venting slot 28C; and a safety cap 29 with a pivot hinge 29A and snap-in recess lock 29B.

The piston is retained within the housing and is movable along the injection axis between a loaded position near the trigger end and an unloaded position near the dispensing end.

The piston comprises a piston head near the dispensing end. The syringe adapter is configured to retain the piston within the housing. For example, the piston head in some embodiments is larger than the opening in the syringe adapter (e.g., formed by the annular portion of the syringe adapter).

The piston head in some embodiments includes a portion sized to extend into the opening of the syringe adapter when the piston is in the unloaded position for contacting the plunger of a needle free syringe.

The compression spring is retained within the housing and around the piston. The compression spring is retained around the piston and is further retained by the piston head proximal to the dispensing end. The interior surface of the housing in some embodiments comprises a portion extending towards the injection axis which blocks the compression spring from contacting the trigger. The piston and compression spring are sized relative to the housing such that the compression spring is compressible along the injection axis within the housing. For example, the outer surfaces of the spring and the piston head in some embodiments slide along the inner surface of the housing.

The configuration of the compression spring in some embodiments is capable of being varied to modify the force in which the piston head will depress the plunger of the needle-free syringe. As a result, it is possible to target different injection depths by choosing different configurations. For example, for subcutaneous injection, the compression spring is capable of being varied depending on the desired depth of penetration. Typical non-limiting examples of spring forces for subcutaneous injection range between 185-10 N to 165-8 N, depending on the degree of fat under the skin. Typical non-limiting examples of spring forces for intramuscular injection are around 200-20 N. Typical non-limiting spring forces for intradermal injection range between 60-5 N and 90-5 N, depending on the age of the subject. The compression spring of various embodiments is capable of thus being configured to provide a spring force of 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245 or 250 N, or greater.

The choice of materials, gauge and coil number to produce a suitable spring to generate these forces is well within the common general knowledge of a person of skill in the art. In one non-limiting example of a spring, the spring material is high carbon spring wire with a wire diameter of 0.4 mm, a mean coil diameter of 3.2 mm, 9 active coils and 11 total coils, a spring stiffness of 0.86 N/mm, and free, working and assembly lengths 25 mm, 19.4 mm and 12.4 mm, respectively. These metrics are merely exemplary, however.

Where the syringe adapter is removable, the housing of some embodiments is capable of being configured to removably accept one or more of alternatively configured syringe adapters, pistons, and springs.

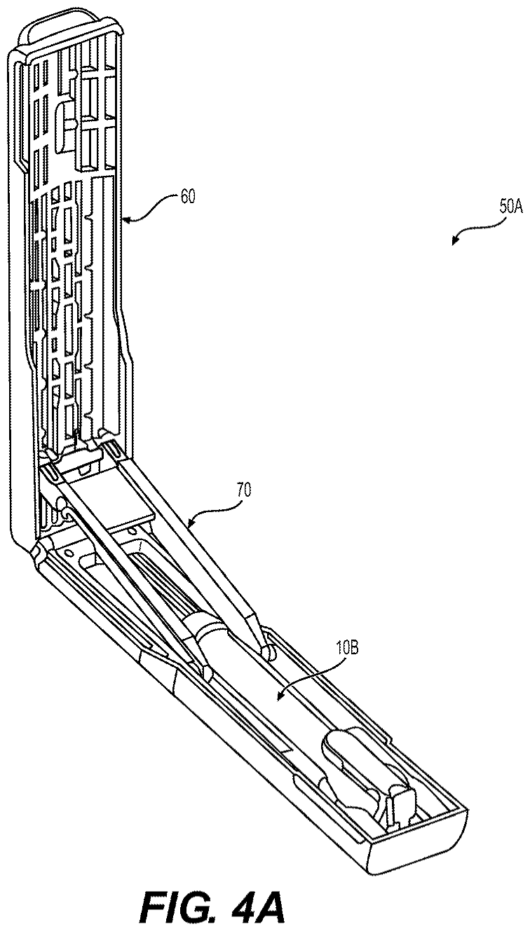

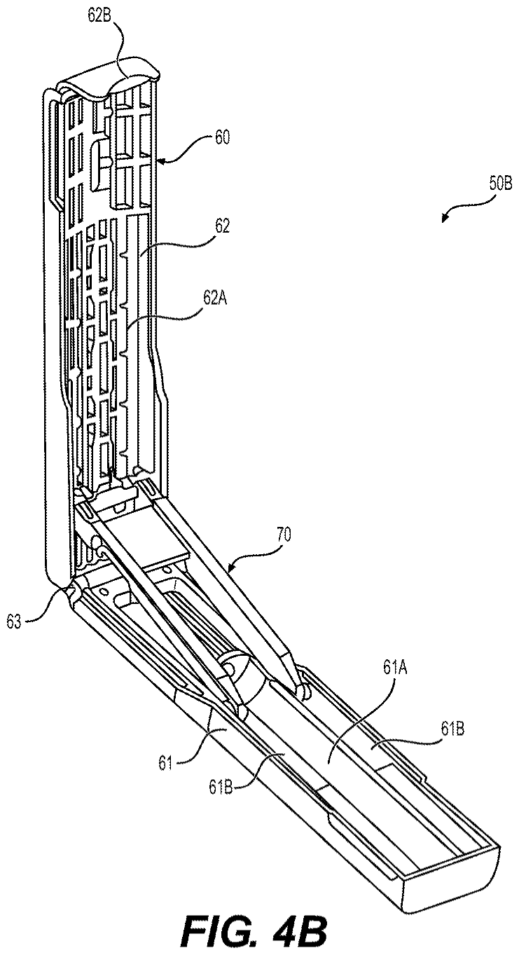

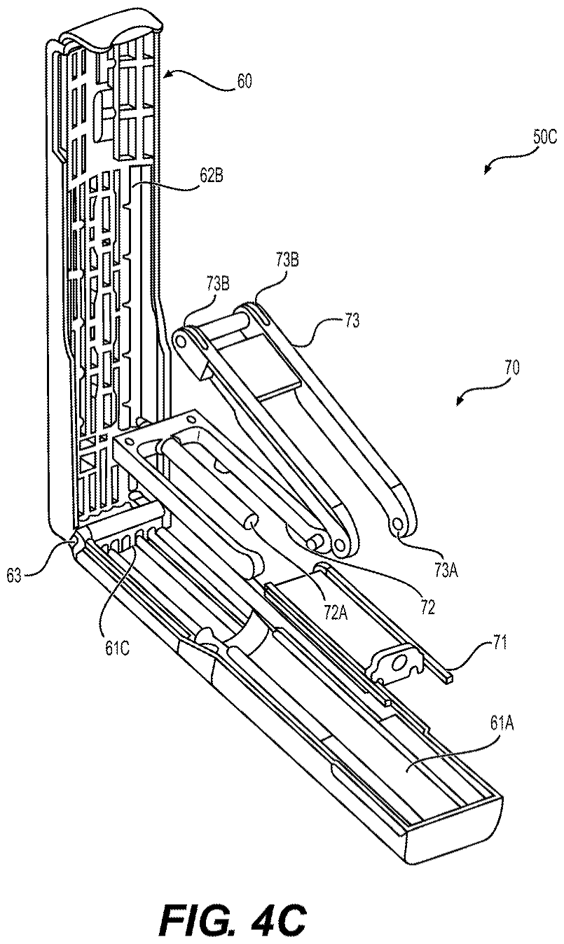

In an exemplary embodiment, the piston is retained in the loaded position by the trigger, which is mounted within the housing near the trigger end and which is movable between a non-actuated position and an actuated position by depression of the push button or lever. The trigger of some embodiments comprises an internal lever biased towards the push lever by a return spring disposed within the housing. The return spring in some embodiments is a clip spring mounted to the interior surface of the housing or elsewhere in the housing. A loader device further facilitates loading of the fluid injection device, as illustrated in FIGS. 4A-4C.

Attention is now turned to FIG. 3B, which, in isometric partial section view, illustrates injector 10B, which is injector 10 in unloaded position. Likewise components labeled likewise. Syringe adapter 30 is shown here in section view with housing tread 31B and syringe socket shoulder 31. The loader is explained next.

Attention is now turned to FIG. 4A, which, in isometric view, illustrates loader assembly 50A with injector 10B positioned to be loaded and lever assembly 60 with loader mechanism 70.

The loader of some embodiments takes the form of a hinged case comprised of top and bottom halves. The loader is movable between an open (as shown) and closed state (not shown). Further details are disclosed in FIGS. 4B and 4C.

Attention is now turned to FIG. 4B, which, in isometric view, illustrates loader compact assembly 50B without injector, readied to load.

Lever assembly 60 comprises lower arm 61, upper arm 62, and arms connector hinge 63. The lower arm has injector receiving socket 61A and sliding guides 61B formed from its body. The upper arm has sliding guide 62 As formed between stiffener ribs. Parts of mechanism 70 slide on said guides.

Attention is finally turned to FIG. 4C, which, in isometric view, illustrates loader disassembled assembly 50C without injector, stripped apart for clarity. Likewise components labeled likewise.

Loading mechanism 70 comprises caddy 71 which is abutted by reaction wall 61C, framed plunger 72 with push pin 72A, forcing arm 73 with hinge 73A and slider tips 73B.

To reduce or eliminate force on the external actuation surface of the fluid injection device during loading, the loader of some embodiments is configured to retain the syringe adapter and prevent movement of the fluid injection device during loading. For example, the syringe adapter of some embodiments comprises an outer surface that extends away from the injection axis beyond the outer surface of the housing and tapers from a wider syringe adapter outer surface proximal end to a narrower syringe adapter outer surface dispensing end. The loader of some embodiments is capable of being configured to receive the fluid injector device on an interior retaining surface comprising a syringe adapter receiver having a rounded, tapered shape corresponding to said syringe adapter outer surface and including a lip adapted to engage the outer surface proximal end of the syringe adapter of the fluid injector device. Since the syringe adapter receiver receives, extends along, and closely fits around the syringe adapter outer surface of the fluid injection device and the lip of the interior retaining surface of the loader is adapted to engage the extended outer surface proximal end of the syringe adapter of the fluid injector device, none of the push lever, safety cap, and external actuation surface are stressed against the interior surface of the loader during loading.

In further embodiments of the present invention, a kit comprises a fluid injector device and a loader. The syringe adapter of the fluid injector device of some embodiments is removable. In some of these embodiments, the kit includes one or more additional compression springs, each of which is distinctly configured to provide a different spring force. Where the syringe adapter is removable, the kit of some embodiments includes one or more additional syringe adapters, each of which is distinctly configured to retain different needle-free syringes, preferably labeled for 0.1-0.5 ml intervals for fluid delivery.

To the skilled in the art of needle free injecting, it shall be obvious that the disclosed and proposed novel features of the invention, including the trigger mechanism, its safety cap, the spring force graduation, the spring loader, and the spring-piston kit, all facilitate safer use, simpler mechanism, lesser cost and more ease and speed of use, thus represent great improvement on the state of art.

The present invention is described above with reference to a preferred embodiment. However, those skilled in the art will recognize that changes and modifications may be made in the described embodiment without departing from the nature and scope of the present invention.

For instance, replacing the coil spring with crest-to-crest wave spring is intuitive and thus instructive as being within the scope of the invention. Crest-to-crest springs, known as Smalley Wave Springs, fit smaller space than coil springs for being half as short for the same force, and allow for the proposed force graduation by wave clipping, which greatly reduces manufacturing cost. Wave springs use flat, not round but rolled from round, wires and, in essence, are a series of leaf springs in contact and continuation around a circular path, fitting into a cylindrical space. Wave springs can be laminated to give damping which reduces recoil felt on the push button or lever of the device.

Furthermore, it is also considered within the scope storing one or more springs, pistons, or spring-piston sets in the loader in sockets or compartments to be handy for the one who needs only a few different spring force settings. That is, it is considered intuitive and hereby instructive to combine the kit with the loader.

Also considered within the scope of the present invention are accessories facilitating the syringe fill-up from a vial and collar sets enlarging the syringe tip area to be pushed against the skin. In some embodiments, a syringe fill-up accessory includes a vial adapter that creates an appropriate transition/connection between the injection nozzle of the syringe and the opening of a vial from which the syringe is to be filled. In some such embodiments, the vial adapter includes three components, a main body, a lid and an insert. In such embodiments the lid covers a syringe fill side of the main body to protect from contaminants. The lid is removable for filling, to expose a retention mechanism on the main body that positively engages with the injection nozzle of the syringe to hold the syringe in proper position during filling. The main body includes a straw that protrudes from the vial-side of the main body, which opposes the syringe fill side of the main body. The straw extends into the vial for filling the syringe. The main body includes an O-ring seal at the top of the straw for engagement with the syringe nozzle. The vial-side of the main body is sized and shaped to engage with the opening of a large vial, as well as with a first end of the vial adapter. The opposing second end of the vial adapter is sized and shaped to engage with the opening of a smaller vial. In operation, the insert is removed from the main body to connect the vial adapter to a large vial. The insert is left connected to the vial adapter to connect the vial adapter to a small vial.

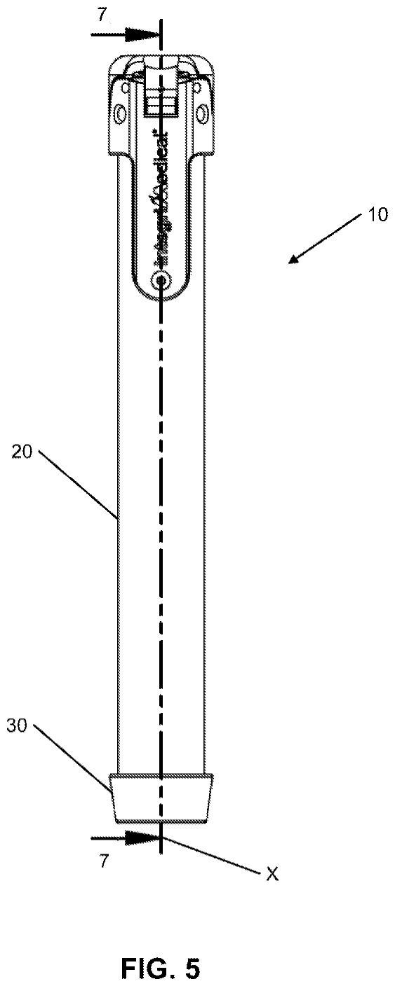

In a further exemplary embodiment of the present fluid injection system and method, the fluid injection device includes a triggering mechanism having a push lever trigger mechanism 126, as illustrated in FIGS. 5-8, rather than a push button. The lever trigger mechanism 126 includes a spring-loaded, user operated lever trigger 126A and an internal extension 126C both pivotable about a trigger mechanism pivot point 126B. In an exemplary embodiment, when a user engages the lever trigger 126A when the piston is loaded, the internal extension 126C of the engagement trigger mechanism 126 is configured to rotate about the trigger mechanism pivot point 126B and apply pressure to an internal, spring-loaded lever of the piston engagement latch 25. The piston engagement latch 25 in this embodiment has the same configuration as in the embodiment shown in FIGS. 1-3B, including being biased by a spring 27 into an engaged position and the internal, spring-loaded lever of the piston engagement latch 25 being connected about an internal pivot point to a piston engagement latch configured to engage with a catch portion of the piston and to hold the piston in a non-actuated position when the piston is loaded.

In this embodiment shown in FIGS. 5-8, the return spring 27 and internal lever of the piston engagement latch 25 are configured to engage with the lever trigger mechanism internal extension 126C and restore the lever trigger 126A to a non-actuated position. When a user applies pressure to the lever trigger 126A and thus the internal extension 126C applies pressure to the piston engagement latch 25 internal lever, the force of the return spring 27 is counteracted, rotating the piston engagement latch 25 about the internal pivot point 25A and releasing the piston head, ultimately allowing the compression spring to expand and fire the piston.

In an exemplary embodiment of the present invention, the lever trigger mechanism pivot point 126B is positioned at the trigger end of the fluid injection device 10 near the internal return spring 27 and the internal spring-loaded lever of the piston engagement latch 25. In an embodiment, the engageable portion of the lever trigger 126A extends outward from the pivot point 126B and is configured for actuation by a user. In an embodiment, the lever trigger 126A extends outward and upward approximately 45-degrees from the pivot point 126B in a loaded, non-actuated position. In an exemplary embodiment, a safety cap 129 opens on the side of the fluid injection device near the trigger lever 126A. In an embodiment, the safety cap 129 opens about a safety cap pivot point near the lever trigger mechanism pivot point 126B.

Such lever trigger mechanism 126 in this embodiment accommodates easier actuation of a trigger compared to a spring-loaded button because it requires less force to actuate a lever about a pivot point compared to a button. Moreover, such lever trigger mechanism allows for utilization of greater spring force compared to prior art needleless injector devices while maintaining an easily engageable trigger for the average user. Furthermore, the present lever trigger mechanism and piston engagement latch design allows for the piston to be latched largely, or about, in line with the piston and the injection axis. This allows for a narrower, sleeker fluid injection device design compared to prior art devices.

In an exemplary embodiment of the present invention, the sound directing construction and lever trigger assembly allow for the present fluid injection device to be easily actuated by laypersons with minimal apprehension. Such device design accommodates use of powerful internal spring force for increased consistency and higher volume injections.

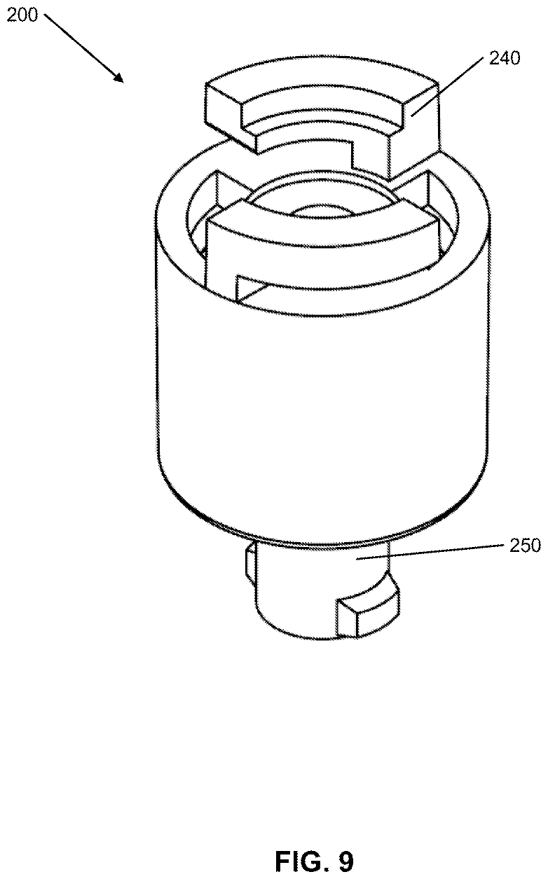

In a further exemplary embodiment of the present invention, the fluid injection system includes a prefilled syringe adapter 200. An exemplary embodiment of a prefilled syringe adapter 200 of the present invention is shown in FIGS. 9-22. In an exemplary embodiment, the prefilled syringe adapter allows a user to aseptically fill a needle-free syringe 40 of the present system with a fluid from a prefilled syringe. Some medications are regularly packaged in prefilled syringes, so the prefilled syringe adapter 200 allows for sterile transfer to a needle-free syringe of the present system for easy use with the fluid injection device 10 of the present invention.

In embodiments, a prefilled syringe adapter 200 creates an appropriate transition or connection between the injection nozzle of a syringe and a prefilled syringe from which the syringe is to be filled. In some such embodiments, the prefilled syringe adapter 200 includes a main body comprising a needle-free syringe side connection 240 configured to engage the dispensing tip and injection nozzle of a needle-free syringe. The adapter main body further includes a prefilled syringe side open connector extension 250. In an exemplary embodiment, the connector extension 250 comprises a luer lock connector configured for engaging with a luer lock prefilled syringe. The prefilled syringe adapter 200 further includes an elongated straw 260 extending through the main body between the needle-free syringe side connector and the prefilled syringe side open connector extension. The adapter needle-free syringe side further comprises an O-ring 220 at the end of the straw for sealing engagement with the needle-free syringe nozzle. In an exemplary embodiment, the prefilled syringe adapter open connector extension 250 and connected straw 260 are sized to receive a prefilled syringe having a needle, the needle fitting within the straw to fill an attached needle-free syringe.

When a needle-free syringe and a prefilled syringe containing a fluid to be injected are connected via a prefilled syringe adapter 200 of the present invention, the plunger of the needle-free syringe can be retracted creating negative pressure, or suction, to fill the needle-free syringe with fluid from the prefilled syringe. Additionally, or instead of retracting the plunger of the needle-free syringe, the plunger of the prefilled syringe can be pushed forward to create pressure to push fluid from the prefilled syringe into the needle-free syringe through the injection nozzle.

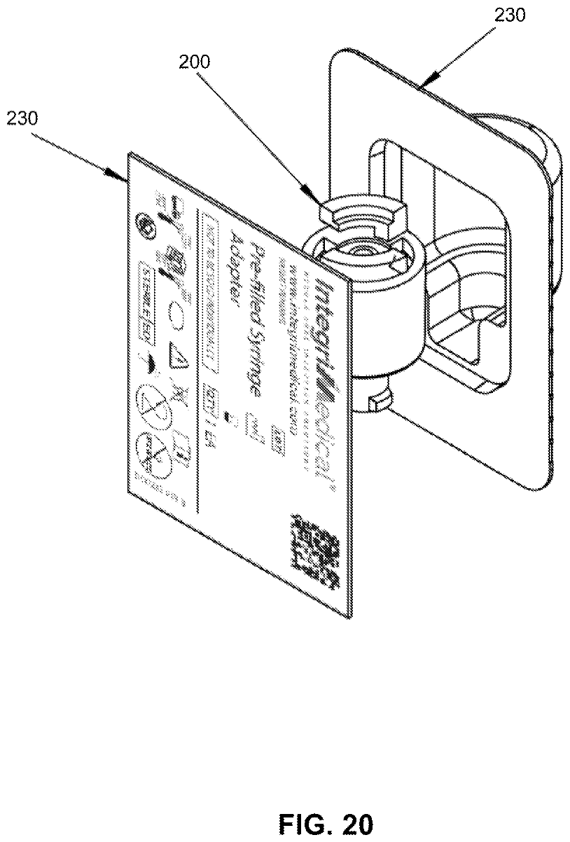

In an exemplary embodiment, the prefilled syringe adapter 200 is packaged in sterile packaging 230, as shown in FIGS. 20-22. Such sterile packaging and use of the prefilled syringe adapter 200 allows for transfer of fluid from a prefilled syringe to a needle-free syringe of the present system without contamination.

In some embodiments, the prefilled syringe adapter further includes a lid configured to cover a needle-free syringe fill side of the main body to protect from contaminants. In such embodiments, the lid is removable for filling.

Certain terminology will be used in the description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology will include the words specifically mentioned, derivatives thereof, and words of similar meaning.

As used in this specification and the appended claims, the singular forms “a”, “an”, and “the” include plural references unless the context clearly dictates otherwise. Thus, for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means ±5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder. “Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

It is also to be understood that the following claims are intended to cover all of the generic and specific features of the invention herein described, and all statements of the scope of the invention which, as a matter of language, might be said to fall therebetween.

Having thus described the invention, what is claimed as new and desired to be secured by Letters Patent is:

1. A fluid injection system, comprising:
a housing comprising an inner surface and an outer surface which extend from a proximal trigger end to a distal dispensing end along an injection axis;
a housing top retainer at said trigger end comprising a lower stop socket, an upper pop-in socket, and a venting slot;
a syringe adapter connected to the dispensing end of the housing and configured to retain a needle-free syringe such that a plunger of the needle-free syringe is movable within the housing along the injection axis;
a piston retained within the housing by the syringe adapter and movable along the injection axis between a loaded position near the trigger end and an unloaded position near the dispensing end, the piston comprising a piston head near the dispensing end and a trigger catch near the trigger end of the housing, the piston head being movably positioned to bias the plunger of the needle-free syringe along the injection axis;
a compression spring associated with the piston, the piston and compression spring sized relative to the housing such that the compression spring is compressible along the injection axis within the housing; and
a trigger mounted within the housing near the trigger end and movable between a non-actuated position and an actuated position by depression of a user trigger means positioned at said trigger end of the housing, the trigger comprising a trigger latch configured to engage the trigger catch when the trigger is in the non-actuated position and the piston is in the loaded position and to release the trigger catch when the trigger is in the actuated position.

2. The fluid injection system of claim 1, wherein the housing, syringe adapter, and needle-free syringe are configured such that a gas within and a sound created within the housing are directed out the venting slot upon actuation of said fluid injection device.

3. The fluid injection system of claim 1, wherein:
the trigger catch comprises an annular engagement surface extending radially outward from the injection axis and facing toward the dispensing end of the housing; and
the trigger latch comprises an engagement surface extending inwardly toward the injection axis and facing toward the trigger end of the housing, and which engages the annular engagement surface of the trigger catch when the trigger is in the non-actuated position and clears the trigger catch when the trigger is in the actuated position.

4. The fluid injection system of claim 1, wherein:
the trigger catch further comprises an annular beveled surface which faces away from the injection axis toward the trigger end of the housing; and
the trigger latch further comprises a beveled surface which faces towards the injection axis and toward the dispensing end of the housing.

5. The fluid injection system of claim 1, wherein said user trigger means comprises a trigger lever rotatable about a trigger lever pivot point.

6. The fluid injection system of claim 5, wherein the trigger further comprises an internal lever biased towards the trigger lever by a return spring disposed within the housing.

7. The fluid injection system of claim 6, wherein:
the trigger further comprises an internal extension means connected to said trigger lever about said trigger lever pivot point; and
said internal extension is configured to counteract said return spring when said trigger lever is engaged.

8. The fluid injection system of claim 6, wherein the return spring is a clip spring biased against the internal lever of the trigger.

9. The fluid injection system of claim 1, further comprising a selectively deployable safety cap for blocking depression of the user trigger means.

10. The fluid injection system of claim 1, wherein the interior surface of the housing comprises a portion extending toward the injection axis configured to block the compression spring from contacting the trigger.

11. The fluid injection system of claim 1, wherein the syringe adapter comprises an outer surface that extends away from the injection axis beyond the outer surface of the housing.

12. The fluid injection system of claim 1, wherein the syringe adapter is removable, and the housing is configured to removably accept alternatively configured syringe adaptors.

13. The fluid injection system of claim 12, wherein:
the piston and the compression spring are removable from the housing when the syringe adapter is removed from the housing; and
the housing is configured to removably accept at least one of the alternatively sized pistons and springs.

14. The fluid injection system of claim 1, wherein the syringe adapter is irremovably affixed to the dispensing end of the housing.

15. The fluid injection system of claim 1, wherein the compression spring is retained around the piston by the piston head.

16. The fluid injection system of claim 1, further comprising:
a prefilled syringe adapter comprising a needle-free syringe connector end opposite a prefilled syringe connector end and a straw extending therethrough; and
wherein said prefilled syringe adapter is configured for sealing connection to a needle-free syringe and a prefilled syringe and to allow aseptic transfer of a fluid from said prefilled syringe into said needle-free syringe.

17. The fluid injection system of claim 16, wherein said prefilled syringe adapter prefilled syringe connector end comprises a luer lock connector configured to engage with a luer lock prefilled syringe.

18. A prefilled syringe adapter device for use with a fluid injection system, the prefilled syringe adapter device comprising:
a main body comprising a needle-free syringe connector end opposite a prefilled syringe connector end and a straw extending therethrough; and
wherein said prefilled syringe adapter device is configured for sealing connection to a needle-free syringe and a prefilled syringe and to allow aseptic transfer of a fluid from said prefilled syringe into said needle-free syringe.

19. The prefilled syringe adapter device of claim 18, wherein said prefilled syringe connector end comprises a luer lock connector configured to engage with a luer lock prefilled syringe.

20. The prefilled syringe adapter device of claim 18, wherein said prefilled syringe connector end and said straw are sized to receive a needled prefilled syringe with a needle of the needled prefilled syringe fitting within the straw to transfer fluid to said needle-free syringe.
